(19) United States
(12) Patent Application Publication
(10) Pub. No.: US 2025/0025887 A1
(43) Pub. Date: Jan. 23, 2025

(54) AIR FILTERING DEVICE FOR AN AIR CONDITIONER

(71) Applicants: MAHLE INTERNATIONAL GMBH, Stuttgart (DE); KARLSRUHER INSTITUT FÜR TECHNOLOGIE, Eggenstein-Leopoldschafen (DE)

(72) Inventors: Andrei BOLOGA, Stutensee (DE); Daniel EBERLE, Ludwigsburg (DE); Sven PETZNER, Welzheim (DE); Hans-Joachim GEHRMANN, Weingarten (DE)

(21) Appl. No.: 18/770,893

(22) Filed: Jul. 12, 2024

(30) Foreign Application Priority Data
Jul. 18, 2023 (DE) 102023206812.5

(51) Int. Cl. B03C 3/12; B03C 3/06; B03C 3/09; B03C 3/41; B03C 3/60; F24F 8/30

(52) U.S. Cl. CPC B03C 3/12; B03C 3/06; B03C 3/09; B03C 3/41; B03C 3/60; F24F 8/30; B03C 2201/06; B03C 2201/08; B03C 2201/30

(57) ABSTRACT

An air filtering device for an air conditioner, in particular for filtering out particles, preferably pollutants, from air is provided. The air filtering device comprises a flow path through which air flows along a flow direction, an ionizer for generating ions in the flow path, and grid structure made of an electrically conductive material, that is upstream of the ionizer in the flow path in the flow direction, which forms a first electrode. A filter is downstream of the ionizer in the flow path in the flow direction which has a layer that forms a second electrode. The air filtering device also comprises a high voltage power source that has an electric pole and an electric counter-pole for generating a high voltage between the pole and the counter pole. The pole is electrically connected to the ionizer. The counter-pole is electrically connected to the first and second electrodes. there is a third electrode near the ionizer 3, electrically separated therefrom, which is electrically connected to the first and second electrodes 5a, 5b.

### CROSS-REFERENCE TO RELATED APPLICATIONS

This application claims priority from German Patent Application No. 102023206812.5, filed Jul. 18, 2023, the entirety of which is hereby incorporated by reference herein.

The invention relates to an air filtering device for an air conditioner.

Modern air conditioners for vehicle interiors typically contain filters with which noxious substances, gases, particulates and unpleasant odors are removed from the air supplied to the vehicle interior.

The high particulate content in the vehicle environment, and thus the air conditioner environment, has proven to be problematic, resulting in high demands to the filtering of particulates from the air that is supplied to the vehicle interior. Filters with a fiber layer are used for filtering out dust particles. Because the space available for these air conditioners is normally limited in modern motor vehicles, there is also comparatively little space for such filters. To ensure that sufficient air can be supplied to the vehicle interior by the air conditioner, these filters must have a low flow resistance, such that pressure losses generated in the air also remain low. For this reason, conventional filters often have an open pore filter fiber layer for removing particles, resulting in a comparatively low mechanical capacity for dust removal on the part of the filter.

To counteract this, the removal of dust particles is improved through electrostatic charging. Unfortunately, the electrostatic charge generated in the production process diminishes over time, such that the filter quickly becomes ineffective with regard to filtering dust.

The object of the present invention is to therefore create new ways of developing air filtering devices. In particular, a better embodiment of such an air filtering device is to be obtained, which makes use of the above approach using an electrostatic charge, and results in an improved removal of particles over the entire service life of the filter.

This problem is solved by the subject matter of the independent claims. Preferred embodiments are the subject matter of the dependent claims.

The fundamental idea of the invention is to place an ionizer containing numerous discharge electrodes—also known to the person skilled in the art as corona discharge electrodes—for generating ions in the flow path, and to place a counter electrode upstream of the ionizer, which is placed in the air flow, electrically separated from the discharge electrodes. This allows an electric field to be generated in the flow path with which particles in the air can be ionized.

The invention proposes electrically connecting a filter that can be electrostatically charged, which is downstream of the discharge electrodes in the ionizer, to the counter electrode that is upstream of the ionizer. This results in another counter electrode downstream of the ionizer in addition to the upstream counter electrode. An electric field is then generated between the discharge electrodes in the ionizer and the counter electrodes, resulting in an improved charging of particles and a polarization effect in the filter.

The invention also proposes placing a counter electrode between the discharge electrodes in the ionizer, which is electrically separated from the discharge electrodes in the flow path. The shape of the field lines in the corona discharge field generated in this manner has a positive effect on the accumulation of charged particles within the electrostatically charged filter, further increasing the polarization effect in the filter material. This maintains and restores the polarization of the filter over time, thus increasing the efficiency of the long-term particle removal, even when the filter is heavily clogged with particles.

This results in an air filtering device that efficiently removes particles, in particular pollutants, from the air in the flow path over the entire service life of the air filtering device, or the filter that is used in the air filtering device.

In detail, the air filtering device according to the invention contains a flow path through which air can flow. There is an ionizer in the flow path, which has an ionizer electrode that can contain numerous discharge electrodes for generating ions therein. There is a grid made of an electrically conductive material upstream of the ionizer in the flow path, which forms a first electrode. The first electrode forms a counter electrode to the discharge electrodes and charges particles upstream of the discharge electrodes. Downstream of the ionizer in the flow path there is a filter that has a layer forming a second electrode. This layer is made of an electrically conductive material, or comprises an electrically conductive material. The filter can preferably be in the form of a boot, thus having a bellows-like form.

The air filtering device also contains a high-voltage power source with an electric pole and counter-pole, and a counter-pole for generating a high voltage between the pole and counter-pole, in particular between −5 kV and −15 kV. A third electrode is then placed between them near the ionizer, electrically separated therefrom, which is electrically connected to the first and second electrodes.

An electric direct, alternating, or impulse corona discharge can then be generated with the ionizer in the air filtering device. An electric potential with negative and positive polarity can be applied to the ionizer, or the ionizer electrode or discharge electrode, for this.

Preferably, however, DC voltage with negative polarity is applied to generate the corona discharge, because a corona discharge with negative polarity results in greater stability and higher values in the corona electron flow than corona discharges with positive polarities. For this reason, the electric pole in the high voltage power source is the negative pole, and the electric counter-pole is positive pole. Alternatively, the electric pole can be positive, and the counter-pole can be negative in the high voltage power source.

In one embodiment of the air filtering device according to the invention the ionizer contains an ionizer electrode for generating the ions in the air flow. The ionizer electrode can also contain numerous discharge electrodes.

In an advantageous embodiment the ionizer electrode can contain two electrode rods, from which at least one electrode tip protrudes, preferably in the form of a needle, toward the first electrode, to generate the ions. The electrode tips preferably extend in the direction opposite the flow direction.

An extremely strong electric field can be generated in this manner near the electrode tip in question, facilitating the generation of a high number of ions by the ionizer. Each electrode tip can form a discharge electrode in the ionizer.

The distance from the grid structure forming the first electrode to the electrode rods in the ionizer is preferably between 20 mm and 50 mm, preferably approx. 25 mm.

The at least two electrode rods can ideally extend in a straight line, transverse, preferably orthogonal, to the flow direction in the flow path. Consequently, the ionizer acts on the entire cross section of the flow path for generating ions.

In another advantageous embodiment, the ionizer electrode can contain at least two, preferably more, preferably parallel electrode rods that are spaced apart from one another. In this embodiment, the third electrode has at least two electrode elements that are spaced apart, each of which are placed between two adjacent electrode rods in the ionizer. This further improves the shape of the field lines in the electric corona field.

At least three electrode rods and at least two electrode elements can preferably alternate in the transverse direction, which is perpendicular to the flow direction, and preferably also perpendicular to the direction of extension.

At least one of the electrode elements can preferably be an electrode plate.

This electrode plate is preferably in a plane that extends in the flow direction. This prevents the electrode plate from causing an excessively strong pressure drop in the air striking the electrode plate or flowing past it.

In another preferred embodiment, the ionizer and third electrode are in a plane that is transverse, preferably orthogonal, to the flow direction.

In another preferred embodiment, at least two, preferably more, particularly all, adjacent electrode rods are spaced apart in a direction orthogonal to the direction of extension, at a distance of 20 mm to 60 mm, preferably 25 mm to 35 mm. This prevents the electrode rods from generating an excessively strong pressure drop in the air flow past.

In an advantageous embodiment, at least two, preferably more, particularly all, adjacent electrode tips can be spaced apart along the direction of extension, at a distance of 1 mm to 30 mm, preferably 5 mm to 9 mm. This prevents the electrode rods from causing an excessively strong pressure drop in the air striking the electrode plate or flowing past it.

The electrode tips are preferably arranged in a grid in the flow path. This results in a better shape of the field lines of the corona field in the flow path.

In another preferred embodiment, at least one of the electrode tips tapers toward the first electrode, particularly preferably in the direction opposite the flow direction, in particular conically. This is preferably the case with numerous, preferably all, of the electrode tips.

In another preferred embodiment, the filter contains a layer through which air can flow, composed of an electrically insulating, in particular dielectric, material for removing particles from the air, which is upstream or downstream of the layer forming the second electrode in the flow path. This electrically insulating, or dielectric, material can be electrostatically charged. This increases the efficiency with which electrically charged particles can be filtered out by the filter. The electrically insulating, or dielectric, material also prevents arc-over from the ionizer to the electrically conductive layer of the filter.

This layer can form a substrate for the layer forming the second electrode. This ensures that the electric corona field passes through the substrate and the ionized particles that are to be filtered out also pass through the second layer. These two layers can be placed on top of one another along the flow direction.

The layer of the filter that forms the second electrode is ideally made of activated carbon, or at least contains activated carbon. This prevents ozone from passing through the filter.

In another advantageous embodiment, the grid structure contains at least two, preferably more, spaced-apart first grid rods, which extend in the air flow transverse, preferably orthogonal, to the flow direction. In this embodiment, the grid structure also contains at least two, preferably more, spaced-apart second grid rods, which also extend in the air flow transverse, preferably orthogonal, to the first grid rods as well as to the flow direction.

The first, second, and third electrodes can preferably be connected to one another. This results in an advantageous shape of the field lines in the electric field for the ionization.

The effect of the polarization of the electric field on the filter element depends on the intensity of the electric field obtained with the electrodes. Higher intensities result in greater polarization effects. Consequently, the corona discharge electrodes should be as close as possible to the entry surface of the filter. In another preferred embodiment, the distance between the ionizer, in particular the electrode rods in the ionizer, to the filter is therefore no more than 30 mm, preferably no more than 7 mm.

The grid structure, or the (first and/or second) rods thereof, is preferably made of steel, ideally stainless steel, or contains such. The ionizer, or ionizer electrodes, can also be made of steel, ideally stainless steel, or contain such, in this variation.

Further features and advantages of the invention can be derived from the dependent claims, drawings, and descriptions in reference to the drawings.

It is understood that the features specified above and described below can be used not only in the given combinations but also in other combinations or in and of themselves without abandoning the framework of the present invention.

Preferred exemplary embodiments of the invention are shown in the drawings, an shall be explained below in greater detail, in which the same reference symbols are used for identical, similar, or functionally identical components.

Therein, schematically:

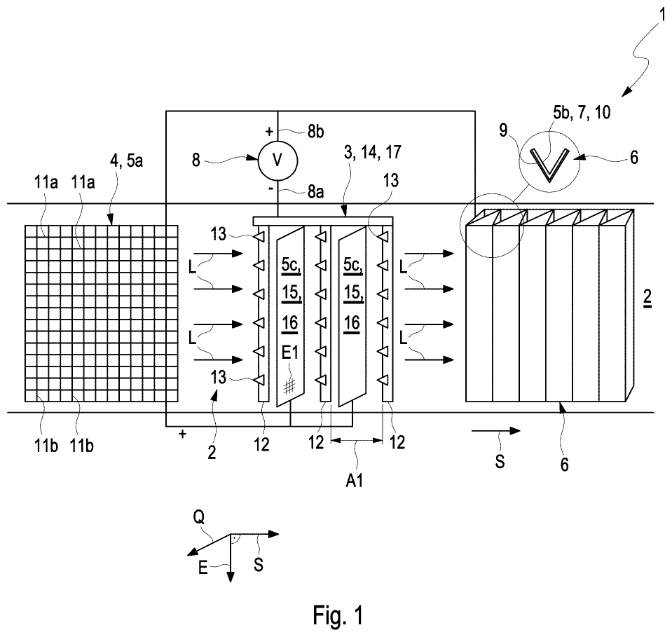

FIG. 1 shows an example of an air filtering device according to the invention in a perspective view,

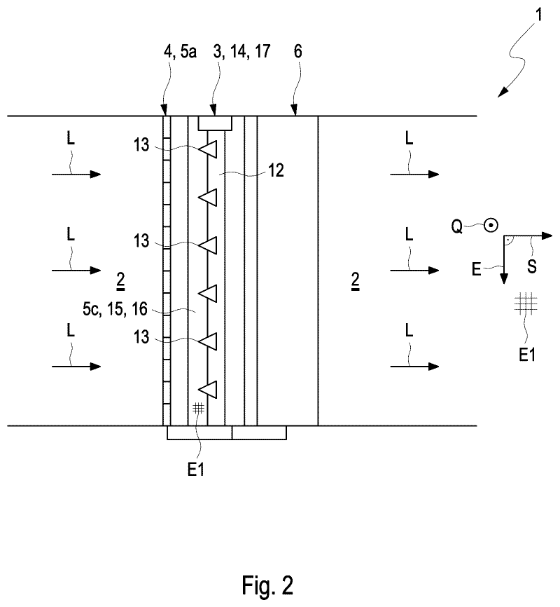

FIG. 2 shows the air filtering device from FIG. 1 in a side view.

FIG. 1 shows a perspective view of an example of an air filtering device 1 according to the invention, FIG. 2 shows a side view thereof. As shown in FIGS. 1 and 2, the air filtering device 1 contains a path 2, along which air L flows in a direction of flow S. The air filtering device 1 also contains an ionizer 3 in the flow path 2 for generating ions. The air filtering device 1 also contains a grid structure 4 made of an electrically conductive material that is upstream of the ionizer 3 in the flow path 2, and forms a first electrode 5a.

As can be seen in FIGS. 1 and 2, the grid structure 4 contains numerous first rods 11a, which are spaced apart and extend in the flow path 2 orthogonally to the flow direction S. The grid structure 4 also contains numerous second rods 11b, which are spaced apart and extend in the flow path 2 orthogonally to the first rods 11a and to the flow direction S. The grid structure 4, or the first and second rods 11a, 11b, can be made of steel, in particular stainless steel. The first and second rods 11a, 11b can form a plane that is perpendicular to the flow direction S.

The air filtering device 1 also contains a filter 6 that is downstream of the ionizer 3 in the flow path 2 in the flow direction S. The filter element 6 also contains a layer 7 that forms a second electrode 5b for the air filtering device 1. This layer 7 is made of an electrically conductive material in this example.

The filter 6 in the example shown in the drawings also contains a layer 9 through which the air L can flow, made of an electrically insulating material for removing particles from the air L. This layer 9 is upstream of the layer 7 forming the second electrode 5b in the flow path 2. The layer 7 of the filter 6 forming the second electrode 6b is formed by activated carbon 10. The two layers 7, 9 of the filter 6 can be layered in the flow direction S.

The air filtering device 1 also contains a high voltage power source 8 with an electric pole 8a and an electric counter-pole 8b for generating a high voltage between the pole 8a and counter-pole 8b. This voltage can be between 5 kV and 15 kV between the pole 8a and counter-pole 8b. The pole 8a is preferably a negative pole, and the counter-pole 8b is preferably positive. This can also be reversed, such that the pole 8a is positive, and the counter-pole 8b is negative.

The negative pole 8a is electrically connected to the ionizer 3 and the positive counter-pole 8b is electrically connected to the first and second electrodes 5a, 5b in FIG. 1. There is also a third electrode 5c near the ionizer 3, electrically separated therefrom, which is electrically connected to the first and second electrodes 5a, 5b. The counter-pole 8b is therefore also electrically connected to the third electrode 5c.

The ionizer 3 has an ionizer electrode 14 in the form of a discharge electrode 17 for generating ions in the flow path 2. The ionizer electrode 14 contains numerous electrode rods 12 spaced apart from one another, which each have electrode tips 13 in the form of needles that extend from the electrode rods 12 toward the first electrode 5a, from which the ions can be generated. The electrode tips 13 form the discharge electrodes 13. The electrode tips 13 protrude away from the respective electrode rods 12 toward the grid structure 4, against the flow direction S.

As FIGS. 1 and 2 show, the electrode tips 13 are arranged in a grid in the flow path 2, and also taper conically toward the first electrode 5a. The ionizer 3, or ionizer electrode 14 and its rods 12, can be made of steel, e.g. stainless steel. The individual rods 12 each extend in the flow path 2 in a straight line in the same direction E, which is orthogonal to the flow direction S. The distance between the electrode rods 12 in the ionizer 3 to the filter 6 is no more than 30 mm, preferably no more than 7 mm.

By way of example, all of the adjacent electrode rods 12 are spaced apart at a distance A1 in a direction that is orthogonal to the direction of extension E, which is 20 mm to 60mm, preferably 25 to 35 mm. In this example, all of the adjacent electrode tips 13 are spaced apart at a distance along the direction of extension E of 1 mm to 30 mm, preferably 5 mm to 9 mm.

As shown in FIGS. 1 and 2, the third electrode 5c has numerous electrode elements 15 that are spaced apart from one another. The individual electrode elements 15 can each form an electrode plate 16. The individual electrode plates 15 are in a plane E1 extending along the flow direction S. Each electrode element 15 from the third electrode 5c is placed between two adjacent electrode rods 12 in the ionizer 4, spaced apart therefrom. The electrode rods 12 and electrode elements 15 thus alternate along the transverse direction Q, which is perpendicular to the flow direction S and also perpendicular to the direction of extension E for the electrode rods 12.

The specification can be readily understood with reference to the following Representative Paragraphs:

Representative Paragraph 1. An air filtering device (1) for an air conditioner, in particular for filtering particles, preferably pollutants, from air (L), containing
a flow path (2) through which air (L) can flow in a flow direction (S),
an ionizer (3), for generating ions in the flow path (2),
a grid structure (4) made of an electrically conductive material, which is upstream of the ionizer (3) in the flow path (2) in the flow direction (S), and forms a first electrode (5a),
a filter (6) downstream of the ionizer (3) in the flow path in the flow direction (S) that has a layer (7) forming a second electrode (5b), comprising an electrically conductive material or made of an electrically conductive material,
a high voltage power source (8) containing an electric pole (8a) and an electric counter-pole (8b), for generating a high voltage (HV), in particular between 5 kV and 15 kV, between the pole (8a) and counter-pole (8b),
wherein the pole (8a) is electrically connected to the ionizer (3), and the counter-pole (8b) is electrically connected to the first and second electrodes (5a, 5b)
wherein there is also a third electrode 5c near the ionizer 3, electrically separated therefrom, which is electrically connected to the first and second electrodes 5a, 5b.

Representative Paragraph 2. The air filtering device according to Representative Paragraph 1, characterized in that the
electric pole (8a) is negative, and the electric counter-pole (8b) is positive in the high voltage power source (8), or
electric pole (8a) is positive, and the electric counter-pole (8b) is negative in the high voltage power source (8).

Representative Paragraph 3. The air filtering device according to Representative Paragraph 1 or 2, characterized in that the ionizer (3) contains an ionizer electrode (14), preferably comprising numerous discharge electrodes (17), for generating ions in the flow path (2).

Representative Paragraph 4. The air filtering device according to any of the Representative Paragraphs 1 to 3, characterized in that the ionizer electrode (14) contains at least two electrode rods (12), spaced apart from one another, from which at least one electrode tip (13), preferably in the form of a needle, protrudes toward the first electrode (5a), to generate the ions.

Representative Paragraph 5. The air filtering device according to Representative Paragraph 4, characterized in that the at least one electrode rod (12) extends in a straight line, in a direction of extension (E) that is transverse, preferably orthogonal, to the flow direction (S) in the flow path (2).

Representative Paragraph 6. The air filtering device according to any of the preceding Representative Paragraphs, characterized in that the third electrode (5c) comprises at least two spaced-apart electrode elements (15), each of which is placed between two adjacent electrode rods (12) in the ionizer (4), spaced apart therefrom.

Representative Paragraph 7. The air filtering device according to Representative Paragraph 6, characterized in that at least three electrode rods (12) and at least two electrode elements (15, 15) alternate along a transverse direction (Q) that is perpendicular to the flow direction (S), and preferably perpendicular to the direction of extension (E).

Representative Paragraph 8. The air filtering device according to Representative Paragraph 6 or 7, characterized in that at least one electrode element (15) forms an electrode plate (16).

Representative Paragraph 9. The air filtering device according to Representative Paragraph 8, characterized in that at least one electrode plate (16) is placed in a plane (E1) that extends along the flow direction (S).

Representative Paragraph 10. The air filtering device according to any of the Representative Paragraphs 4 to 9, characterized in that at least two, preferably more, particularly all, adjacent electrodes (12) are at a spacing (A1) to one another in a direction orthogonal to the direction of extension (E), which is 20 mm to 60 mm, preferably 25 mm to 35 mm.

Representative Paragraph 11. The air filtering device according to any of the Representative Paragraphs 4 to 10, characterized in that at least two, preferably more, particularly all, adjacent electrode tips (13) are at a spacing to one another along the direction of extension (E), which is 1 mm to 30 mm, preferably 5 mm to 9 mm.

Representative Paragraph 12. The air filtering device according to any of the Representative Paragraphs 4 to 11, characterized in that the electrode tips (13) are arranged in a grid in the flow path (2).

Representative Paragraph 13. The air filtering device according to any of the Representative Paragraphs 3 to 12, characterized in that at least one, preferably more, particularly all, of the electrode tips (13) tapers toward the first electrode (5a), in particular conically.

Representative Paragraph 14. The air filtering device according to any of the preceding Representative Paragraphs, characterized in that the filter (6) contains a layer (9) through which air can flow, made of an electrically insulating material for removing particles from the air (L), which is upstream or downstream of the layer (7) forming the second electrode (5b) in the flow path (2).

Representative Paragraph 15. The air filtering device according to Representative Paragraph 14, characterized in that the layer (7) of the filter (6) forming the second electrode (5b) is made of activated carbon (10) or at least contains activated carbon (10).

Representative Paragraph 16. The air filtering device according to Representative Paragraph 14 or 15, characterized in that the two layers (7, 9) of the filter (6) are layered on top of one another along the flow direction (S).

Representative Paragraph 17. The air filtering device according to any of the preceding Representative Paragraphs, characterized in that
the grid structure (4) comprises at least two, preferably more, first rods (11a) that are spaced apart from one another, which extend in the flow path (2) in a direction transverse, preferably orthogonal, to the flow direction (S),
the grid structure (4) comprises at least two, preferably more, second rods (11b) that are spaced apart from one another, which extend in the flow path (2) in a direction transverse, preferably orthogonal, to both the first rods (11a) and the flow direction (S).

Representative Paragraph 18. The air filtering device according to any of the preceding Representative Paragraphs, characterized in that the distance between the ionizer (3), in particular the electrode rods (12) in the ionizer (3), and the filter (6) is no more than 30 mm, preferably no more than 7 mm.

Representative Paragraph 19. The air filtering device according to any of the preceding Representative Paragraphs, characterized in that the first, second and third electrodes (5a, 5b) are electrically connected to one another.

Representative Paragraph 20. The air filtering device according to any of the preceding Representative Paragraphs, characterized in that
the grid structure (4), or the (first and/or second) rods (11a, 11b), is made of steel, in particular stainless steel, or contains steel, and/or
the ionizer (4), or the ionizer electrode (14), is made of steel, in particular stainless steel, or contains steel, in particular stainless steel.

1. An air filtering device for an air conditioner, in particular for filtering particles, preferably pollutants, from air, comprising
a flow path through which air can flow in a flow direction,
an ionizer, configured to generate ions in the flow path,
a grid structure formed from of an electrically conductive material, which is upstream of the ionizer in the flow path in the flow direction, and forms a first electrode,
a filter downstream of the ionizer in the flow path in the flow direction, the filter comprises that has a layer forming a second electrode, comprising an electrically conductive material or made of an electrically conductive material,
a high voltage power source comprising an electric pole and an electric counter-pole, for generating a high voltage between 5 kV and 15 kV, between the pole and counter-pole,
wherein the pole is electrically connected to the ionizer, and the counter-pole is electrically connected to the first and second electrodes
further comprising a third electrode near the ionizer 3, electrically separated therefrom, the third electrode is electrically connected to the first and second electrodes.

2. The air filtering device according to claim 1, wherein the
electric pole is negative, and the electric counter-pole is positive in the high voltage power source, or
electric pole is positive, and the electric counter-pole is negative in the high voltage power source.

3. The air filtering device according to claim 1, wherein the ionizer contains an ionizer electrode, comprising numerous discharge electrodes, for generating ions in the flow path.

4. The air filtering device according to claim 1, wherein the ionizer electrode comprises contains at least two electrode rods (12), spaced apart from one another, from which at least one electrode tip protrudes toward the first electrode, to generate the ions.

5. The air filtering device according to claim 4, wherein the at least one electrode rod extends in a straight line, in a direction of extension that is transverse to the flow direction in the flow path.

6. The air filtering device according to claim 1, wherein the third electrode comprises at least two spaced-apart electrode elements, each of which is placed between two adjacent electrode rods in the ionizer, spaced apart therefrom.

7. The air filtering device according to claim 6, wherein at least three electrode rods and at least two electrode elements alternate along a transverse direction that is perpendicular to the flow direction.

8. The air filtering device according to claim 6, wherein at least one electrode element forms an electrode plate.

9. The air filtering device according to claim 8, wherein at least one electrode plate is placed in a plane that extends along the flow direction.

10. The air filtering device according to claim 4, wherein at least two adjacent electrodes are at a spacing to one another in a direction orthogonal to the direction of extension, which is 20 mm to 60 mm.

11. The air filtering device according to claim 4, wherein at least two adjacent electrode tips are at a spacing to one another along the direction of extension, which is 1 mm to 30 mm.

12. The air filtering device according to claim 4, wherein the electrode tips are arranged in a grid in the flow path.

13. The air filtering device according to claim 3, wherein at least one of the electrode tips tapers toward the first electrode, in particular conically.

14. The air filtering device according to claim 1, wherein the filter comprises layer (9) through which air can flow, formed from an electrically insulating material for removing particles from the air, which is upstream or downstream of the layer forming the second electrode in the flow path.

15. The air filtering device according to claim 14, wherein the layer of the filter forming the second electrode is formed from or contains activated.

16. The air filtering device according to claim 14, wherein the two layers of the filter are layered on top of one another along the flow direction.

17. The air filtering device according to claim 1, wherein
the grid structure comprises at least two first rods that are spaced apart from one another, which extend in the flow path in a direction transverse to the flow direction,
the grid structure comprises at least two second rods that are spaced apart from one another, which extend in the flow path in a direction transverse to both the first rods and the flow direction.

18. The air filtering device according to claim 1, wherein the distance between the ionizer, in particular the electrode rods in the ionizer, and the filter is no more than 30 mm.

19. The air filtering device according to claim 1, wherein the first, second and third electrodes are electrically connected to one another.

20. The air filtering device according to claim 1, wherein
the grid structure, or the first rod and/or the second rod are formed from or contain steel, and/or
the ionizer, or the ionizer electrode, is formed from or contains steel.

21. The air filtering device of claim 5, wherein the at least one electrode rod extends orthogonal to the flow direction in the flow path.

22. The air filtering device of claim 4, wherein the at least one electrode is in the form of a needle.

23. The air filtering device of claim 7, wherein the at least two electrode elements are perpendicular to the direction of extension.

24. The air filtering device of claim 10, wherein the at least two adjacent electrodes are at a spacing to one another of 25 mm to 35 mm.

25. The air filtering device of claim 11, wherein the adjacent electrode tips are at a spacing of 5 mm to 9 mm.

26. The air filtering device of claim 17, wherein the at least two first rods extend orthogonal to the flow direction, and
the at least two second rods extend orthogonal to the first rods and the flow direction.

27. The air filtering device of claim 18, wherein the distance between the electrode rods in the ionizer and the filter is no more than 7 mm.
